(19) United States
(12) Patent Application Publication
(10) Pub. No.: US 2025/0268560 A1
(43) Pub. Date: Aug. 28, 2025

(54) METHOD FOR LIVER DISEASE DIAGNOSIS AND GRADING

(71) Applicants: The Regents of the University of California, Oakland, CA (US); Virginia Polytechnic Institute and State University, Blacksburg, VA (US)

(72) Inventors: Dorathy Tamayo-Murillo, Bonita, CA (US); Claude Sirlin, La Jolla, CA (US); Michael Andre, La Jolla, CA (US); Aiguo Han, Blacksburg, VA (US)

(21) Appl. No.: 19/063,299

(22) Filed: Feb. 26, 2025

Related U.S. Application Data
(60) Provisional application No. 63/557,995, filed on Feb. 26, 2024.

(51) Int. Cl. A61B 8/08; A61B 8/00; G16H 10/40; G16H 50/30

(52) U.S. Cl. CPC A61B 8/08; A61B 8/463; A61B 8/469; A61B 8/5261; G16H 10/40; G16H 50/30

(57) ABSTRACT

A method for quantitatively assessing fat of a liver is disclosed. The method comprises: obtaining measured ultrasound data comprising an ultrasound image of the liver and corresponding radio frequency (RF) data of the liver; identifying a field of interest (FOI) in the ultrasound image; determining, for each of a plurality of graphical elements that are within the FOI, values of one or more parameters of interest that are indirect measures of the fat in the liver using the RF data and a pre-defined relationship; and enabling assessment of the fat in the liver based on the determined values.

### CROSS-REFERENCE TO RELATED APPLICATIONS

This patent document claims priorities to and benefits of U.S. Provisional Patent Application No. 63/557,995 entitled “METHOD FOR LIVER DISEASE DIAGNOSIS AND GRADING” filed on Feb. 26, 2024. The entire content of the aforementioned patent application is incorporated by reference as part of the disclosure of this patent document.

### TECHNICAL FIELD

This patent document relates to techniques to assess liver fat using quantitative ultrasound (QUS).

### BACKGROUND

Non-invasive, accurate, precise, accessible and affordable methods to assess liver fat in primary care settings are needed but none are currently available. Techniques for providing such assessments of liver fat are highly desirable.

### SUMMARY

Disclosed herein are devices, systems and methods that pertain to non-invasive, accurate, precise, independent, accessible and affordable techniques to assess liver fat using QUS with a point-of-care ultrasound (POCUS) device that are not device specific.

In one example aspect, a method for quantitatively assessing fat of a liver is disclosed. The method can include: obtaining measured ultrasound data comprising an ultrasound image of the liver and corresponding radio frequency (RF) data (e.g., RF signals) of the liver; identifying a field of interest (FOI) in the ultrasound image; determining, for each of a plurality of graphical elements that are within the FOI, values of one or more parameters of interest that are indirect measures of the fat in the liver using the RF data and a pre-defined relationship; and enabling assessment of the fat in the liver based on the determined values.

In another example aspect, a system for quantitatively assessing fat of a liver is disclosed. The system can include: a point-of-care ultrasound (POCUS) device configured to obtain ultrasound data comprising an ultrasound image of the liver and corresponding radio frequency (RF) data of the liver; and one or more processors configured to receive the ultrasound data from the POCUS device and to: identify a field of interest (FOI) in the ultrasound image, determine, for each of a plurality of graphical elements that are within the FOI, values of one or more parameters of interest that are indirect measures of the fat in the liver using the RF data (e.g., RF signals from the FOI) and a pre-defined relationship, generate a liver fat deposition map by assigning, to each of the graphical elements, a color associated with the values, and enable display of the liver fat deposition map to allow assessment of the fat in the liver based on the determined values.

In another example aspect, a computer program product having code stored thereon is disclosed. The code, when executed by a processor, can cause the processor to implement a method for quantitatively assessing fat of a liver, comprising: obtaining measured ultrasound data comprising an ultrasound image of the liver and corresponding radio frequency (RF) data of the liver; identifying a field of interest (FOI) in the ultrasound image; determining, for each of a plurality of graphical elements that are within the FOI, values of one or more parameters of interest that are indirect measures of the fat in the liver using the RF data (e.g., RF signals from the FOI) and a pre-defined relationship; and enabling assessment of the fat in the liver based on the determined values.

In another example aspect, a computer system that includes one or more computing platforms may be configured to implement any of the above-described methods.

In yet another aspect, any of the above-described method may be embodied in the form of computer-executable code and stored on a storage medium.

These, and other, features and aspects are further disclosed in the present document.

### BRIEF DESCRIPTION OF DRAWINGS

FIG. 1 is a schematic showing an example embodiment based on the disclosed technology and some examples of current clinical practices and their associated limitations.

FIG. 2 shows an example hardware platform that can be implemented with embodiments of the disclosed techniques.

FIGS. 3A-3F show example results obtained in a study performed in accordance with embodiments of the disclosed techniques.

FIGS. 4A-4F show additional example results obtained in a study performed in accordance with embodiments of the disclosed techniques.

FIGS. 5A-5C show additional example results obtained in a study performed in accordance with embodiments of the disclosed techniques.

FIG. 6 shows a flowchart of an example method based on embodiments of the disclosed technology.

### DETAILED DESCRIPTION

Metabolic dysfunction-associated steatotic liver disease (MASLD, formerly known as non-alcoholic fatty liver disease) is a silent disease with an estimated global prevalence of 32%. MASLD encompasses a spectrum of abnormalities, ranging from abnormal liver fat accumulation to cirrhosis. It is a risk factor for hepatocellular carcinoma, cancer in other organs, and premature death. Screening in primary-care settings for MASLD is urgently needed to enable early diagnosis and timely lifestyle modifications, which can stabilize or reverse the condition, but there are no accurate and affordable non-invasive tools for this purpose in such settings.

Available options for MASLD screening are not ideal or widely accessible. Blood-based laboratory tests such as alanine aminotransferase and aspartate aminotransferase, while widely available, are neither accurate nor precise. Liver biopsy, the current reference standard for MASLD diagnosis, is poorly suited due to cost, risk of serious complications, sampling error, and limited availability. Chemical shift encoded magnetic resonance imaging (CSE-MRI)-based proton density fat fraction (PDFF) is non-invasive, accurate, and precise, but has limited access and contraindications that reduce its effectiveness.

Radiologist-interpreted conventional B-mode ultrasound is a common imaging tool for MASLD screening given its safety, patient tolerance and affordability, but is subjective and imprecise. To overcome the limitations of conventional ultrasound, QUS methods have been developed to measure liver tissue parameters numerically and provide more objective assessment. Such parameters include the controlled attenuation parameter, which is measurable with a relatively costly device utilized mainly in specialist clinics, and various QUS parameters measurable on advanced cart-based ultrasound systems, which may be device-dependent, are not available at point-of-care and require operational expertise. Accurate, affordable, and accessible tools to screen for MASLD in primary-care settings are needed but not yet available. QUS implemented on a handheld device is a potential avenue to address this unmet need.

The disclosed embodiments, among other features and benefits, provide an affordable, accessible, and accurate alternative to available technologies for the diagnosis and grading of liver fat. Embodiments disclosed herein offer better affordability compared to MRI or U.S. Food and Drug Administration cleared large clinical ultrasound systems, including but not limited to Ultrasound-Guided Attenuation Parameter (UGAP), Controlled Attenuation Parameter (CAP), which are device-dependent, or radiologist interpreted images. Furthermore, the disclosed embodiments are non-invasive, avoid biopsy, and correlate with the gold standard MRI PDFF. Techniques disclosed herein can be deployed as a cheap screening tool for fatty liver disease diagnosis, grading, and potential monitoring.

The present patent application discloses some embodiments that can be implemented into systems for providing a diagnostic of liver fat.

Some disclosed embodiments relate to methods of providing a diagnostic of liver fat. For example, one method uses quantitative ultrasound data from a handheld device such as a POCUS device. The method may include obtaining raw radio frequency data from the POCUS device, assessing multiple ultrasound parameters, and using them to grade and diagnose fatty liver disease.

Some disclosed embodiments relate to methods to quantify fat and provide a diagnostic in an optimal manner using raw data/biomarkers. In the current state of the art, the data acquired from a POCUS device is typically altered to create a B-mode grayscale image. In one example method disclosed herein, the method includes replacement of typically acquired grayscale B-mode images with newly developed colorized quantitative ultrasound images (e.g., liver fat deposition maps) overlayed on B-mode images. Among other features and benefits, these colorized images can be easy to conceptualize by primary care physicians and patients and can later be used for health education.

Some disclosed embodiments may be implemented using a handheld POCUS device that retains the ability to display B-mode images.

Various aspects of the disclosed embodiments are discussed herein.

In one aspect, the disclosed embodiments relate to expanding the technologic capacity of existing POCUS technologies and presenting data in a quantifiable way that may be used for fat quantification and diagnosis. Embodiments disclosed in the present patent document can expand the capabilities of an existing commercial handheld POCUS device and utilize the data for fat quantification and diagnosis.

In another aspect, the disclosed embodiments relate to eliminating interobserver variability in the diagnosis and grading of MASLD. For example, radiologists' interobserver variability (current clinical practice) can be eliminated by applying methods disclosed herein, thereby increasing accuracy of diagnosis. This can improve current clinical practice.

In another aspect, new users of the methods and systems disclosed herein can be minimally trained operators rather than a radiologist or sonographer. Options of new users include but are not limited to a primary care physician, nurse, or medical trainee.

In another aspect, the disclosed embodiments relate to increased access to disease diagnosis, grading, and monitoring. For example, techniques disclosed herein have a high likelihood to deliver a new capability to end-users to increase access of imaging for early diagnosis. Given that MASLD is a global epidemic, the disclosed embodiments can be made widely accessible in low-resourced communities, which do not have an on-site sonographer or radiologist. Additionally, devices disclosed in the present patent document are affordable, which increases the chance of wide implementation/increased access, as does the device's small size and minimal maintenance cost.

In another aspect, the disclosed embodiments have high likelihood of commercial availability and shortened time to implementation and dissemination.

In another aspect, the disclosed embodiments may change the status quo of imaging utilization. The status quo as it pertains to the non-invasive clinical monitoring of MASLD is to utilize B-mode imaging, CAP, or MRI. MRI is accurate but limited by cost and accessibility. CAP is limited by variability and a high failure rate in obese patients, although it is suboptimal it is utilized in hepatology clinics because of its relative affordability and accessibility, similar barriers with UGAP that is device-specific. Embodiments disclosed herein may outperform CAP and UGAP in accuracy, cost, and accessibility. These technologic advances can replace B-mode imaging, CAP, and UGAP for the assessment of liver fat and eliminate the need for biopsy. These advancements can also facilitate a means for the implementation of an affective screening strategy for MASLD.

In another aspect, the disclosed embodiments relate to changing the clinical and research paradigm to early diagnosis and prevention. The disclosed embodiments may shift the current research paradigm away from MRI and conventional ultrasound at a tertiary care or specialty clinic level and expand it to the community clinic and primary care level. By bringing this technology to the primary care level it will move the clinical and research paradigm to early prevention of MASLD and Metabolic Dysfunction-Associated Steatohepatitis (MASH), instead of focusing on late prevention of more advance disease such as fibrosis and HCC.

Certain implementations of the disclosed embodiments may be used as a global screening strategy for the fatty liver disease epidemic. Embodiments can be used in a primary care or research setting. New visual images can be used for patient education.

One example method to provide a quantitative assessment of liver fat involves obtaining ultrasound data of a liver of a subject. The ultrasound data may comprise an ultrasound image and corresponding radio frequency (RF) data of the liver. In some embodiments, the ultrasound data is obtained using a POCUS device. The POCUS device may be commercially available. In some implementations, the POCUS device is handheld and the image is obtained by a user upon activation of the handheld device (e.g., via the push of a button located on the handheld device). The user may be a novice with minimal training or a trained operator, such as a sonographer or radiologist. The POCUS device may be implemented with additional forms of equipment, such as hardware, software, or other types of data acquisition circuitry. In some implementations, the POCUS device with the additional equipment can be used to obtain the image of the liver in an automated fashion. The RF data may be transmitted, in a wired or wireless manner, to one or more computing devices (e.g., personal computer, data processor, etc.) for processing. The RF data may be processed, e.g., using an algorithm (a mathematical model or pre-defined relationship as described elsewhere in the present document), to determine various tissue parameters of interest from the RF data that are indirect measures of liver fat. These parameters can include, for example, fundamental QUS parameters such as attenuation coefficient, backscatter coefficient, Lizzi-Feleppa slope, Lizzi-Feleppa intercept, Kappa, and Mu, or combinations thereof. The example method may include making an assessment of the fat in the liver based on the determined values. One or more pre-defined relationships may also be used to determine the parameters of interest and to make the assessment. In one example, the pre-defined relationship is determined based on a measured ground truth data and sample ultrasound data, where the measured ground truth data and the sample ultrasound data are obtained from a plurality of sample subjects. In some implementations, the plurality of sample subjects corresponds to a population of subjects of a predetermined age range, weight range, or other characteristics. For each sample subject, the measured ground truth data may include a distribution of liver fat metrics (e.g., values of PDFF at various locations within the liver of the sample subject) that are related to the various parameters of interest. The ground truth data may include, but is not limited to, MRI data, one or more MRI images, biopsy data, or numerical values of liver fat (e.g., PDFF). In some implementations, the example method involves providing results of processing the RF data (e.g., the parameters of interest) on the device display or to a different device for display in real-time. Such results may also include a quantification of liver fat, or other forms of information associated with liver fat.

In some implementations, the example method can be used to provide a diagnostic of the liver related to inflammation, fibrosis, or volume.

In some example embodiments, a pre-defined relationship can be applied to different subgroups of subjects. In some other embodiments, different pre-defined relationships may be developed for different subgroups, e.g., different age groups, different ethnicity groups, different groups organized based on pre-existing conditions, etc.

Some disclosed embodiments involve generating one or more images or parameter maps showing fat deposition in the liver. In one example method, a two-dimensional (2D), colorized liver fat deposition map is generated using ultrasound data, comprising an ultrasound image of a liver and corresponding RF data of the liver, obtained from a POCUS device in accordance with disclosed techniques. The method may include identifying a field of interest (FOI) in the ultrasound image and determining, for each of a plurality of graphical elements that are within the FOI, values of one or more parameters of interest that are indirect measures of the fat in the liver using the RF data and a pre-defined relationship. The pre-defined relationship may be determined based on a measured ground truth data and sample ultrasound data as previously described. In some implementations, the parameters of interest are QUS parameters. Each of the graphical elements may be assigned a color associated with the values of the one or more parameters of interest, or combinations of the values of the one or more parameters of interest, such that the liver fat deposition map is a colorized map.

In some embodiments, each pixel in the liver fat deposition map is registered to a graphical element in the ultrasound image. The graphical element may be a pixel or voxel. Each pixel in the liver fat deposition map is also associated with values of one or more parameters of interest (e.g., QUS parameters) obtained from processing the RF data as disclosed in this patent document. Each pixel in the liver fat deposition map is assigned a color associated with a value of a specific parameter of interest or values of a combination of parameters of interest.

In some embodiments, the colorized liver fat deposition map is a three-dimensional (3D) map comprising a plurality of voxels calibrated to a volumetric image (e.g., 3D ultrasound image) of the liver. Each voxel in the 3D map is registered to a voxel in the volumetric image and is associated with one or more of the parameters of interest (e.g., QUS parameters) obtained from processing the RF data as disclosed in this patent document. Each voxel in the 3D map may be assigned a color associated with a value of a specific parameter of interest or values of a combination of the parameters of interest.

In some implementations, a value of the fat in the liver is determined based on the values of the one or more parameters of interest associated with the graphical elements of a region in the liver fat deposition map. In some implementations, the 2D or 3D liver fat deposition maps may be provided to a display. For example, the liver fat deposition map may be superimposed over an image of the liver (e.g., a B-mode grayscale image, 2D or 3D ultrasound image, MRI image, etc.) such that the deposition of fat in the liver, values of the parameters of interest, and/or values of fat in the liver can be visualized over associated regions in the image.

In another example method, a colorized liver fat deposition map is generated using ultrasound data and ground truth MRI data of the liver. In this example method, the ultrasound data includes an ultrasound image of the liver and associated RF data of the liver obtained in accordance with disclosed techniques. Using one or more processors, the ultrasound image is gridded into a plurality of subdivisions within an identified FOI. Each graphical element within each subdivision of the ultrasound image is associated with values of one or more QUS parameters obtained by processing the RF data as disclosed in the present patent application. The graphical elements of each subdivision are assigned to graphical elements of a liver fat deposition map. The graphical elements in the liver fat deposition map are also assigned a color associated with the values of the one or more QUS parameters, or the values of combinations of the one or more QUS parameters. The graphical elements of the ultrasound image and the liver fat deposition map may be pixels or voxels.

In some implementations, the values of the QUS parameters associated with each subdivision are averaged to obtain average QUS parameters for each subdivision. The average QUS parameters for each subdivision can be registered to graphical elements in a corresponding region of a ground truth MRI image or map, where each graphical element in the MRI image or map is associated with a liver fat estimate as measured from MRI. The average QUS parameters, within subdivisions or within a predetermined FOI, can be compared to average liver fat estimates in a corresponding region of the MRI image by calibrating the subdivisions or the FOI to the graphical elements in the MRI image or map.

One advantage of colorized liver fat deposition maps is the ability to illustrate the spatial heterogeneity of various parameters of interest in the liver, aiding patients and clinicians to understand how liver fat may be changing within the liver over time.

Some disclosed embodiments may involve automatically reporting results obtained from processing RF data acquired using a POCUS device as disclosed in the present patent document. These results (e.g., QUS parameters, liver fat content metrics, liver fat distribution maps, etc.) may be shared to a computer or processor such that the results may be automatically entered into a medical report of a patient via the computer or processor.

Some disclosed embodiments relate to obtaining raw RF data from a POCUS device. One example system includes a POCUS device configured to obtain raw RF data associated with the liver without acquiring an ultrasound image of the liver. The raw RF data can be processed in accordance with methods disclosed in the present patent document to obtain various parameters of interest related to liver fat, allowing for real-time reporting of such parameters. In one example, upon activation of the POCUS device, the raw RF data may be transmitted to a computing device and processed to obtain QUS parameters which can then be provided to a display or announced. In some implementations, the POCUS device or another device in communication with the POCUS device may provide an alert when values of any of the parameters of interest, or combinations thereof, exceed a predetermined threshold.

In some example embodiments, the processing of RF data obtained from a POCUS device is based on a mathematical model determined from measured ground truth data and sample ultrasound data obtained from a plurality of sample subjects. The measured ground truth data is obtained for each subject and may include an MRI image of the liver of the subject and associated MRI data of the liver of the subject. The sample ultrasound data is obtained at areas corresponding to regions in the MRI image. The measured ground truth data for each subject may comprise a distribution of liver fat metrics (e.g., values of PDFF) in the liver of the subject, where each liver fat metric in the distribution has a known relationship to QUS parameters. In some example embodiments, such as the example methods previously described, the measured ground truth data and the sample ultrasound data are used to determine the one or more pre-defined relationships.

The pre-defined relationship(s) may be represented using a mathematical model. In some embodiments, the mathematical model is device-independent, functioning across various point-of-care ultrasound (POCUS) systems used to obtain the radiofrequency (RF) data. In some embodiments, the mathematical model includes one or more device-specific or device-model specific values of parameters that may provide improved accuracy. In one example, the mathematical model is developed using reference QUS parameters obtained from ultrasound and MRI data acquired from a population of sample subjects. By determining relationships between at least two of the reference QUS parameters, the mathematical model can be derived and applied to online analysis of RF data acquired by any commercially available POCUS device in accordance with techniques disclosed herein. The mathematical model can be used to determine various liver fat metrics (e.g., fat fraction). In some implementations, the mathematical model is based on reference parameters obtained from a population of subjects of a predetermined age range, weight range, or other characteristic.

Methods described herein (e.g., the method 600) may be embodied in the form of a computer program product or computer-executable code stored on a storage medium. For example, the computer program product or the computer-executable code may include an analytics package capable of real-time processing RF data obtained from a POCUS device. Results generated by the analytics package may include a colorized map or image, a QUS parameter value, a QUS parameter classification (normal, abnormal, below or above a threshold, the presence of a liver condition), an automated alert that can be delivered through, e.g., text, visual, or audio signals when a liver condition is detected, or the like, or a combination thereof, as described in the present patent document. The computer program product or the computer-executable code could, for example, be stored on a local machine or downloaded by technologies of different commercial ultrasound vendors.

Some disclosed techniques may be implemented as part of a system for providing an assessment of liver fat.

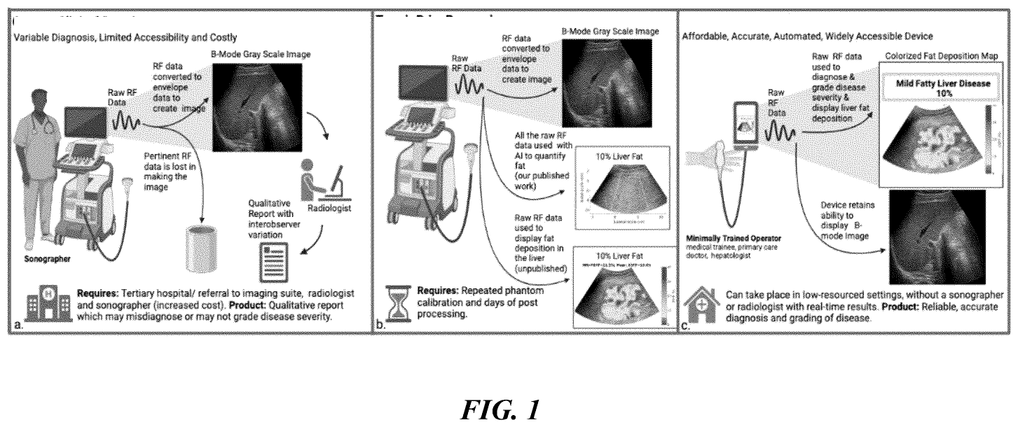

FIG. 1 shows an example embodiment based on the disclosed technology and some examples of current clinical practices and their associated limitations for liver diagnosis (e.g., variability in diagnosis, limited accessibility, device dependency and cost limitations). As shown in FIG. 1 (left), in many clinical settings raw RF data is acquired via ultrasound by a sonographer. Some of the RF data is converted to envelope data to create a B-mode gray scale image while another portion of the RF data is lost in the process of making the gray scale image. The gray scale image is then analyzed by a radiologist to generate a qualitative report. However, the qualitative report is subject to interoperator and interobserver variation that may result in misdiagnosis or errors in grading disease severity. Additionally, current clinical techniques require a hospital or other imaging suite setting, a radiologist, and a sonographer, all of which come with added costs. FIG. 1 (center) shows another existing technique for diagnosing liver fat. In this technique, all the raw RF data obtained from the ultrasound measurement is used to quantify fat (e.g., using artificial intelligence (AI)). The raw RF data is also used to generate an image displaying fat deposition in the liver. However, the aforementioned technique is limited by requiring repeated phantom calibration and hours to days of post processing. FIG. 1 (right) shows a part of an example system based on the disclosed technology and some of the benefits of the system in comparison to existing techniques. The example system shown in FIG. 1 (right) includes a handheld device which may be implemented using additional equipment or circuitry (unpictured) included in the system. The handheld device can be used to obtain raw RF data (e.g., at the push of a button located on the device). The raw RF data can be processed in accordance with disclosed techniques to diagnose and grade disease severity and to generate a colorized liver fat distribution map for display (top box in FIG. 1 right). The liver fat distribution map may include an indicator related to a liver fat estimate or other diagnostic parameter at one or more locations on the map. The handheld device may also retain the ability to display B-mode images and can be implemented by a minimally trained operator (e.g., medical trainee, primary care doctor, hepatologist). Compared to existing techniques, the disclosed system shown on right in FIG. 1 can be implemented in low-resourced settings, without a sonographer or radiologist, and can provide results in real-time, enabling reliable and accurate diagnosis and grading of disease.

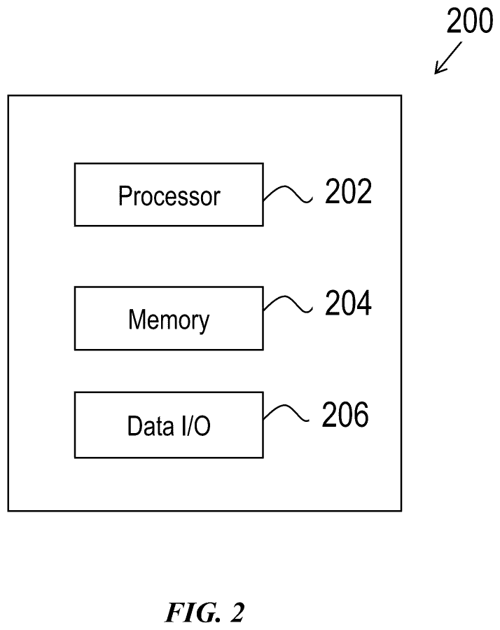

FIG. 2 shows an example hardware platform 200. One or more such platforms 200 may be used to implement a system or method described herein. In various embodiments, the platforms 200 may be used for a distributed computing system or may correspond to computing sources located in a computing cloud.

The platform 200 may include one or more processors 202. The processors 202 may be configured to execute code. The platform 200 may include one or more memories 204 for storage of code, data and intermediate results of execution. The platform 200 may include one or more interfaces 206 for data input or output. For example, the interfaces 206 may be a network connection such as a wired Ethernet or wireless Wi-Fi connection or may be communication ports such as USB, and the like. Various techniques described in the present patent document may be implemented in a cloud-based computing system where multiple hardware platform 200 may be present.

In the description that follows, an example study, which was performed in accordance with disclosed techniques, is described.

The purpose of the study was to demonstrate the feasibility and examine the performance of QUS using a handheld POCUS device, in accordance with the disclosed techniques, for liver fat assessment in adults with overweight or obesity, using contemporaneous PDFF as reference. The study focused on adults with excess adiposity because they are technically challenging to image and a clinically relevant population for MASLD screening. PDFF was as reference because it is well accepted as the most accurate and precise non-invasive quantitative biomarker for diagnosis and quantification of liver fat.

Regarding the study design and participants, the example study was ancillary to an ongoing prospective longitudinal observational study (“the parent study”). Inclusion criteria for the parent study included: adults (≥18 years old) enrolled in a standard-of-care bariatric surgery program and scheduled for gastric bypass or sleeve gastrectomy, class 2 or 3 obesity (BMI≥35 kg/m2) at time of study referral, and willingness to participate. Exclusion criteria for the parent study included: MRI contraindication(s), pregnancy, girth or weight exceeding scanner capacity, bleeding diathesis, known liver malignancies, regular and excessive alcohol consumption within 2 years prior to recruitment, clinical or laboratory evidence of liver disease other than MASLD, current use of steatogenic and/or hepatotoxic medications. Participants of the parent study underwent MRI visits at several time points before and after weight loss surgery (usually sleeve gastrectomy); MRI was performed with estimation of liver fat as described later.

For this example study, participants of the parent study were recruited between March 2022 and August 2022. Inclusion criteria were enrollment in the parent study and willingness to participate. There were no exclusion criteria. Participants in the example study underwent a single-day research ultrasound visit contemporaneously to one of the parent study MRI visits (target: same day, window: up to one month). During the ultrasound visit, QUS was performed using a handheld POCUS device by two operators, as described later.

The study endpoints were diagnostic accuracy for hepatic steatosis (defined as PDFF≥5%), correlation with contemporaneous PDFF, and inter-operator (expert versus novice) reproducibility of handheld QUS.

Research coordinators collected demographic and laboratory data as part of the parent study on the day of MRI. FIB-4 levels (which provide likelihood scores for presence of hepatic fibrosis) were calculated from age, platelets, alanine aminotransferase, and aspartate aminotransferase. Height and weight for the example study were collected on the day of QUS. Body mass index (BMI) was calculated. Participants were instructed to fast for >2 hours prior to both MRI and ultrasound.

PDFF exams were performed to estimate liver fat. PDFF values from segments 5-8 were averaged to estimate the overall fat from the right lobe.

In the example study, ultrasound exams were performed using a POCUS device (Butterfly IQ+ handheld ultrasound probe, Butterfly Network Inc, Burlington, MA). This battery-powered device utilizes a two-dimensional linear array of 9,000 capacitive micromachined ultrasonic transducers (CMUTs) on a single silicon chip, a newer transducer technology. It is designed to operate through a wired USB-C connection to an Apple iOS cellphone or tablet running the Butterfly application. In the exemplary experimental setup, the Butterfly IQ+ was connected to laptop computer (Apple Powerbook) running a proprietary Butterfly software development kit (SDK version 1.15), which enabled the recording of B-mode images and the corresponding beam-formed RF ultrasound data. In conventional US, the backscattered ultrasound pressure waves from the tissue are converted by the transducer to 1D RF signals of voltage vs. time for each element, which are then assembled into beamformed 2D frames of RF data capturing the entire field of view. The beamformed RF data are then processed to generate B-mode (gray scale) images, after which the RF data are discarded. In QUS, the RF data are saved and analyzed to yield quantitative estimates of ultrasound properties.

In the example study, the POCUS operators included two diagnostic registered medical sonographers, each with over 10 years of sonography experience, and two novice ultrasound operators. The novices were a first-year research resident and a clinical research coordinator, neither with any prior ultrasound scanning experience. Each operator underwent one training session using the Butterfly device on a liver tissue-mimicking phantom and one training session on a human volunteer. Training sessions were supervised by an experienced ultrasound medical physicist and a technician from Butterfly Network Inc.

Participants underwent two POCUS exams at each ultrasound visit, one exam performed by one of the two expert sonographers and the other performed by one of the two novice operators. The selection of operators for each exam was based on personnel availability. For each participant, the exam by the expert sonographer was performed first and the exam by the novice operator afterward. As this was a feasibility study, the order was not randomized.

POCUS exams were performed with participants in the left lateral decubitus position with their right arm abducted. Five sets of transverse images of the right liver lobe were acquired through an intercostal space, using a standard setting for abdominal imaging available on the Butterfly device (“Deep Abdomen”). Each set was obtained in a separate 5-to 10-second breath-hold and comprised two consecutive button presses. The first button press recorded a B-mode image and the second button press captured the corresponding RF data. Using the same system and setting, one set of images was then obtained of the reference phantom.

In performing quantitative ultrasound analysis, the B-mode images and RF data captured on the Butterfly device were analyzed off-line to compute six QUS parameters as shown in Table I below. These include two fundamental power spectral QUS parameters (attenuation coefficient and backscatter coefficient: AC and BSC), two backscatter-derived parameters (Lizzi-Feleppa [LF] slope and LF intercept), and two statistical parameters derived from the RF signal envelope (kappa and mu). BSC was log transformed to yield 10log₁₀BSC with units of dB (henceforth, logBSC). For each QUS parameter, all available measurements per exam were averaged to yield a single value. For illustrative purposes, parametric maps were generated showing QUS parameter values overlain on the corresponding B-mode image.

TABLE I
Exemplary QUS Measured Parameters

| Type of QUS | QUS | |
|---|---|---|
| parameter | parameter | Description |
| Fundamental | Attenuation | Marker of US energy loss. Reflects composition |
| spectral | coefficient | |
| parameters | (AC) | |
| | Backscatter | Marker of “echogenicity”. Reflects tissue |
| | coefficient | microstructure. Usually log transformed |
| | (BSC) | |
| Parameters | Lizzi-Feleppa | Slope from linear regression of 10log10 (BSC) vs |
| derived | slope | US frequency |
| from BSC | Lizzi-Feleppa | Intercept from linear regression of 10log10 (BSC) |
| | intercept | vs US frequency |
| Envelope | Kappa | Ratio of coherent to incoherent backscattered |
| statistical | | signal. Reflects tissues microstructure |
| parameters | | disorganization |
| | Mu | Number of scatters per resolution cell. Reflects |
| | | scatter density |

Note.-
Kappa and Mu are obtained by fitting a homodyned K distribution to the RF signal envelope

The B-mode images and RF data were analyzed using an open-source software tool implemented on a computer. An image analyst under the supervision of a radiologist and a physicist placed a polygonal FOI manually on each B-mode image of the right lobe captured using the Butterfly device with the SDK. The FOI was drawn to capture as much representative liver parenchyma as possible while avoiding liver edges, shadows, and other artifacts. No effort was made to avoid blood vessels, as prior work showed that excluding blood vessels did not improve QUS performance for assessing liver fat. The software automatically propagated the polygonal field of interest from the B-mode image to corresponding spatially mapped RF data. A bioengineering graduate student under the supervision of an experienced bioengineering professor then computed the six QUS parameters summarized in Table I from the spatially mapped RF data. AC and BSC were computed using a reference phantom method, described elsewhere, between 1.7 and 2.6 MHz, a bandwidth around the center frequency of the received RF signals (i.e., best signal-to-noise ratio). BSC was log transformed to yield logBSC as described earlier. LF slope and intercept were obtained by using linear regression of logBSC against frequency. Kappa and mu were computed assuming a homodyned K distribution. This distribution was originally suggested by others to model the statistics of laser speckle in turbulent media and adopted by some to model ultrasound echo envelope signals. Alternative distribution models (e.g., Rayleigh, Rician, K) are special cases of homodyned K. While the above analysis was performed by a user, in some implementations, the analysis may be carried out in an automated fashion (e.g., using one or more computing devices).

Using receiver operating characteristic (ROC) analysis, the performance of each QUS parameter for classifying hepatic steatosis (defined as PDFF≥5%) was assessed. The assessment was made for expert and novice QUS measurements separately. Areas under the ROC curves (AUCs) were computed with DeLong 95% confidence intervals (CIs). Using expert QUS measurements, a classification cutoff was selected for each QUS parameter to maximize Youden's index. The expert-measurement derived cutoffs were applied to estimate the sensitivity, specificity, total accuracy, and corresponding exact binomial 95% CIs of each parameter, separately for experts and novices

Scatterplots of QUS parameters acquired with POCUS versus contemporaneous PDFF were generated in the example study. Spearman rank correlations were computed between each QUS parameter and PDFF, for expert and novice QUS measurements separately. Cubic smoothing splines were placed on each plot to help visualize the shape of the relationships. Also computed were the Spearman rank correlations between QUS parameters acquired with a full-size ultrasound system by expert sonographers with contemporaneous PDFF using similar data from another study performed elsewhere.

To examine reproducibility of each QUS parameter between expert and novice operators, Bland-Altman (BA) plots were used, as well as precision metrics recommended by the Quantitative Imaging Biomarker Alliance (QIBA): Bland-Altman bias and its significance, 95% limits of agreement (LOA), intra-class correlation coefficient (ICC), reproducibility coefficient (RDC, computed as one half of the width of the LOA), and within-subject coefficient of variation (wCV).

As the example study was a feasibility study, no cross-validation was applied nor adjusting for multiple comparisons. Additionally, the analyses were constrained to successfully acquired data without an intention-to-diagnose framework.

The example study enrolled 18 participants (17 female) with characteristics summarized in Table II below. Mean age, body mass index (BMI), and right-lobe PDFF were 43 +14 years, 33±3 kg/m², and 6±4% (range, 2-14%), respectively. Eight of 18 (44%) participants had hepatic steatosis as defined previously. Median interval between MRI and US exams was 6 days (range, 0-31 days).

TABLE II
Cohort Characteristics in the Example Study
Demographics

| Age | 43 ± 14 years | (range, 22 to 62 years) |
|---|---|---|
| Sex | | |
| Female | 17 | (94%) |
| Male | 1 | (6%) |
| Race | | |
| White | 11 | (61%) |
| Black or African American | 1 | (6%) |
| Asian | 0 | (0%) |
| Native American | 0 | (0%) |
| Native Hawaiian or other Pacific Islander | 1 | (6%) |
| Other | 6 | (17%) |
| Not reported | 2 | (11%) |

Ethnicity

| | | |
|---|---|---|
| Latino/Hispanic | 8 | (44%) |
| Non-Latino/Hispanic | 7 | (39%) |
| Other | 1 | (6%) |
| Not reported | 2 | (11%) |

Anthropometrics

| | | |
|---|---|---|
| Height | 165 ± 8 cm | (range, 152 to 185 cm) |
| Weight | 91 ± 10 kg | (range, 74 to 105 kg) |
| Body mass index (BMI) | 33 ± 3 kg/m² | (range, 28 to 39 kg/m²) |
| Obesity (BMI > 30 kg/m²) | 15 | (88%) |
| Overweight or obese (BMI > 25 kg/m²) | 18 | (100%) |
| Waist circumference | 104 ± 10 cm | (range, 81 to 121 cm) |

Labs

| Aspartame aminotransferase (AST) | 24 ± 9 U/L | (range, 14 to 47 U/L) |
|---|---|---|
| Alanine aminotransferase (ALT) | 23 ± 12 U/L | (range, 155 to 391 U/L) |
| Platelets Mean ± SD, (range) × 1000/mcL | 273 ± 70 × 1000/mcL | (range, 155 to 391 × 1000/mcL) |
| FIB-4 Score Category | | |
| <1.3 (low likelihood of advanced fibrosis) | 14 | (78%) |
| 1.3-2.67 (intermediate likelihood of advanced | 4 | (22%) |
| fibrosis) | | |
| >2.67 (high likelihood of advanced fibrosis) | 0 | (0%) |

Study design elements

| | | |
|---|---|---|
| Days after weight-loss surgery | 145 ± 121 days | (range, 8 to 399 days) |
| Days between MRI and POCUS | Median: 6 days | (range, 0 to 31 days) |

MRI

| | | |
|---|---|---|
| PDFF results right lobe (%) | 6 ± 4 | (2-14) |
| Steatosis (PDFF ≥ 5%) | 8 | (47%) |

QUS parameter values (obtained by expert operators)

| | | |
|---|---|---|
| AC | 0.6 ± 0.1 dB/cm-MHz | (range, 0.4 to 0.8 dB/cm-MHz) |
| logBSC | −38.4 ± 5.3 dB | (range, −45.6 to −27.5 dB) |
| Lizzi-Feleppa slope | 0.93 ± 2.2 dB/MHz | (range, −5.6 to 2.2 dB/MHz) |
| Lizzi-Feleppa intercept | −36.4 ± 8.4 dB | (range, −47.6 to −17.5 dB) |
| Kappa (unitless) | 0.73 ± 0.1 | (0.6 to 0.9) |
| Mu (unitless) | 6.1 ± 1.4 | (4.2 to 9.0) |

In the example study, expert sonographers successfully captured at least one B-mode image and its corresponding set of RF data in 17 of 18 participants. One sonographer had one failed exam (no successful RF acquisitions from the liver) due to rapid sequential button presses for all five acquisitions that resulted in overwriting the data. Novice operators successfully captured at least one B-mode image and the corresponding set of RF data in all 18 participants. Novice operators failed to record the contemporaneous phantom acquisition on two occasions, each time due to rapid sequential button presses with overwriting of the data. During data analysis, the missing phantom data were recovered by utilizing the reference phantom data acquired with identical settings by a novice operator on a different day (this was justified as repeated QUS measurements of the phantom made with the POCUS device remained stable for over 6 months; data not shown). In every case in which RF data were acquired, all six QUS parameters were computed successfully.

The analyses discussed below were performed using successfully acquired or recovered RF data (n=17 participants for expert sonographers, n=18 participants for novice operators)

Five QUS parameters provided good to excellent accuracy for steatosis classification: AC, logBSC, LF Intercept, Kappa and Mu; among these five, AUC ranged from 0.94 to 0.97 for expert sonographer measurements and from 0.88 to 0.94 for novice operator measurements. At Youden-based cutoffs derived from expert sonographer exams, these five parameters provided sensitivities ranging from 0.86 to 1.00 for expert sonographers and from 0.63 to 1.00 for nonexpert operators, and specificities from 0.8 to 1.0 for expert sonographers and 0.5 to 1.0 for nonexperts. LF Slope provided modest accuracy for steatosis classification: AUC was 0.74 (95% CI 0.49-1.00) for expert sonographers and 0.74 (95% CI 0.48-0.99) for novice operators, with sensitivities and specificities at Youden-based cutoffs ranging from 0.43 to 1.0. Results are summarized in Table III, along with accuracy parameters achieved with logBSC acquired by a physician using a full-size system, as reported elsewhere.

TABLE III
Diagnostic Accuracy of each QUS Parameter for Classification
of Fatty Liver in the Example Study

| | | | | | | Total |
|---|---|---|---|---|---|---|
| | | AUC | | Sensitivity | Specificity | Accuracy |
| Parameter | Operator | (CI) | Cutoff | (CI) | (CI) | (CI) |
| AC | Expert | 0.94 | 0.70 | 0.86 | 1.00 | 0.94 |
| | | (0.82-1.00) | dB/cm- | (0.42-1.00) | (0.69-1.00) | (0.71-1.00) |
| | Novice | 0.91 | MHz | 0.75 | 0.70 | 0.72 |
| | | (0.78-1.00) | | (0.34-0.97) | (0.34-0.93) | (0.47-0.90) |
| logBSC | Expert | 0.96 | −36.4 | 0.86 | 1.00 | 0.94 |
| | | (0.86-1.00) | dB | (0.42-1.00) | (0.69 1.00) | (0.71-1.00) |
| | Novice | 0.88 | | 0.63 | 1.00 | 0.83 |
| | | (0.65-1.00) | | (0.25-0.92) | (0.69-1.00) | (0.59-0.96) |
| LF slope | Expert | 0.74 | −2.57 | 0.43 | 1.00 | 0.77 |
| | | (0.49-1.00) | dB/MHz | (0.10-0.82) | (0.69-1.00) | (0.50-0.93) |
| | Novice | 0.738 | | 0.63 | 0.80 | 0.72 |
| | | (0.48-0.99) | | (0.25-0.91) | (0.44-0.98) | (0.47-0.90) |
| LF intercept | Expert | 0.96 | −36.4 | 0.86 | 1.00 | 0.94 |
| | | (0.86-1.00) | dB | (0.42-1.00) | (0.69-1.00) | (0.71-1.00) |
| | Novice | 0.88 | | 0.75 | 0.80 | 0.79 |
| | | (0.70-1.00) | | (0.35-0.97) | (0.44-0.98) | (0.52-0.94) |
| Kappa | Expert | 0.96 | 0.76 | 0.86 | 1.00 | 0.94 |
| | | (0.86-1.00) | | (0.42-1.00) | (0.69-1.00) | (0.71-1.00) |
| | Novice | 0.93 | | 1.00 | 0.70 | 0.83 |
| | | (0.78-1.00) | | (0.63-1.00) | (0.35-0.93) | (0.59-0.96) |
| Mu | Expert | 0.97 | 6.25 | 1.00 | 0.90 | 0.94 |
| | | (0.91-1.00) | | (0.59-1.00) | (0.56-1.00) | (0.71-1.00) |
| | Novice | 0.94 | | 1.00 | 0.50 | 0.72 |
| | | (0.83-1.00) | | (0.63-1.00) | (0.19-0.81) | (0.47-0.90) |
| log BSC on full-size | Expert | 0.95 | −24.0 | 0.87 | 0.910 | 0.88 |
| system, another | | (0.90-1.00) | dB | (0.77-0.94) | (0.75-0.98) | (0.80-0.94) |
| study 2015 | | | | | | |

Note.-
Another publication, described elsewhere, presented the result for the untransformed BSC. To enable a direct comparison, we converted the original threshold of 0.0038 to logBSC threshold of −24.02. This is an order-preserving transformation which would not affect the results of the threshold search or the performance parameters. LF = Lizzi-Feleppa.

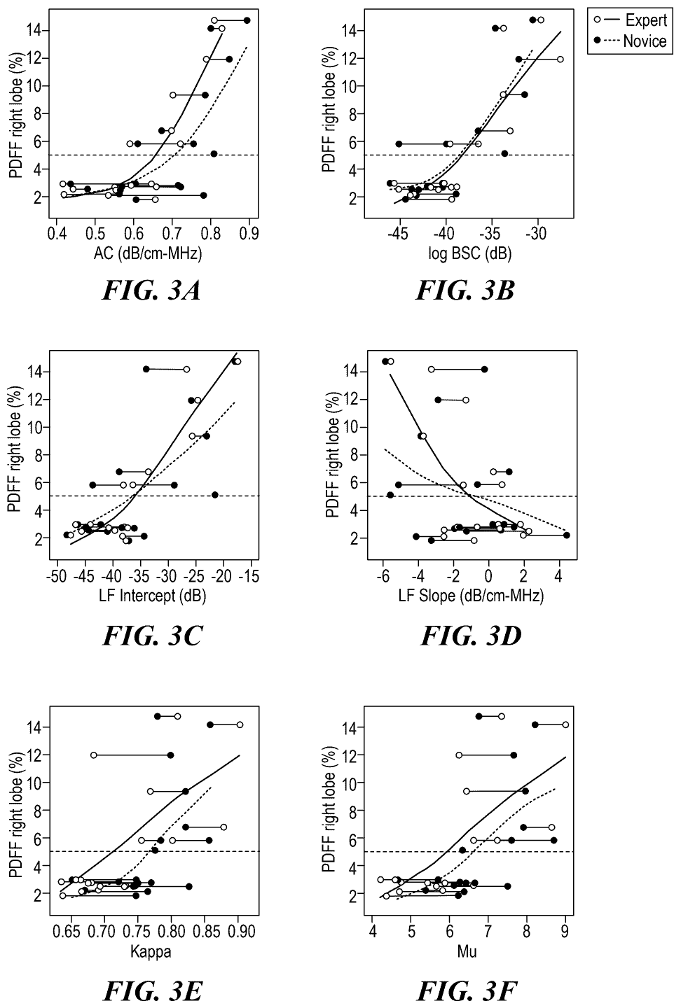

FIGS. 3A-3F show example scatterplots of PDFF vs. each QUS parameter: AC (FIG. 3A), logBSC (FIG. 3B), LF intercept (FIG. 3C), LF slope (FIG. 3D), Kappa (FIG. 3E), and Mu (FIG. 3F). On each plot, expert and novice measurements are marked, respectively. Expert and novice measurements for the same participants are connected by a thin horizontal line. Non-horizontal lines on the plot are trend lines, generated by smoothed cubic splines, and intended to illustrate the overall shape of the relationship between PDFF and QUS measurements by operator type. The horizontal dashed line is the 5% PDFF threshold for the diagnosis of hepatic steatosis. The relationship between one or more QUS parameter and MRI PDFF may be used as a reference standard for quantitatively assessing fat of a liver based on ultrasound data acquired using, e.g., a POCUS, in a clinical setting. The results from the assessment may be further used to diagnose the presence of disease and disease severity/disease grading.

Five QUS parameters correlated positively and significantly with PDFF: AC, logBSC, LF Intercept, Kappa, and Mu; among these five, Spearman's rho ranged from 0.62 to 0.72 for expert sonographers (all P values ≤0.0012) and from 0.53 to 0.69 for novice operators (all P values ≤0.023) as shown in FIGS. 3A-3F. LF Slope correlated negatively, weakly, and non-significantly with PDFF: rho was −0.43 for expert sonographers (P=0.09) and −0.22 for novice operators (P=0.38) measurements. Results are summarized in Table IV below, along with the Spearman rank correlations achieved with a full-size system by expert sonographers, recomputed from a study performed elsewhere.

TABLE IV
Correlation Coefficients Between Quantitative US Parameters
and MRI Proton Density Fat Fraction in the Example Study

| | EXPERT with full-size | | | | | |
|---|---|---|---|---|---|---|
| QUS | system, another | | NOVICE | | EXPERT | |
| Parameter | study 2020 | | with POCUS | | with POCUS | |
| vs. PDFF | (n = 102) | | (n = 18) | | (n = 17) | |
| AC | 0.65, | (p < .001) | 0.64 | (p < .005) | 0.72 | (p < .005) |
| logBSC | 0.74 | (p < .001) | 0.69 | (p < .005) | 0.70 | (p < .005) |
| LF slope | −0.07 | (p = 0.52) | −0.22 | (p = 0.38) | −0.43 | (p = 0.09) |
| LF | 0.60 | (p < .001) | 0.53 | (p < .05) | 0.71 | (p < .005) |
| intercept | | | | | | |
| Kappa | 0.65 | (p < .001) | 0.59 | (p < .01) | 0.62 | (p < .01) |
| Mu | 0.63 | (p < .001) | 0.63 | (p < .01) | 0.65 | (p < .005) |

Note.-
shown are Spearman rank rho correlations (with p-values)
AC = attenuation coefficient. BSC = backscatter coefficient. LF = Lizzi-Feleppa. POCUS = point-of-care ultrasound

Bland-Altman (BA) metrics from the example study are summarized in Table V below.

TABLE V
Inter-operator Reproducibility in the Example Study

| | Bias* | | Limits of agreement* | ICC | CV % | RDC* |
|---|---|---|---|---|---|---|
| AC | −0.05 ± 0.12 | (p = 0.13) | −0.29-0.19 | 0.54 | 15.0 | 0.24 |
| logBSC | 1.28 ± 3.7 | (p = 0.17) | −5.97-8.53 | 0.73 | 6.8 | 7.25 |
| LF slope | 0.39 ± 2.29 | (p = 0.49) | −4.09-4.88 | 0.57 | NA | 4.49 |
| LF intercept | 0.43 ± 4.57 | (p = 0.70) | −8.52-9.38 | 0.86 | 8.7 | 8.95 |
| Kappa | −0.05 ± 0.06 | (p < .01) | −0.16-0.07 | 0.54 | 7.2 | 0.12 |
| Mu | −0.74 = 1.01 | (p < .01) | −2.71-1.23 | 0.59 | 14.2 | 1.97 |

AC = attenuation coefficient. BSC = backscatter coefficient. LF = Lizzi-Feleppa, RDC = inter-operator (expert vs. novice) reproducibility coefficient. NA = not applicable
*units for AC, logBSC, LF slope, and LF intercept are dB/cm-MHz, dB, dB/MHz, and dB, respectively. Kappa and mu are unitless

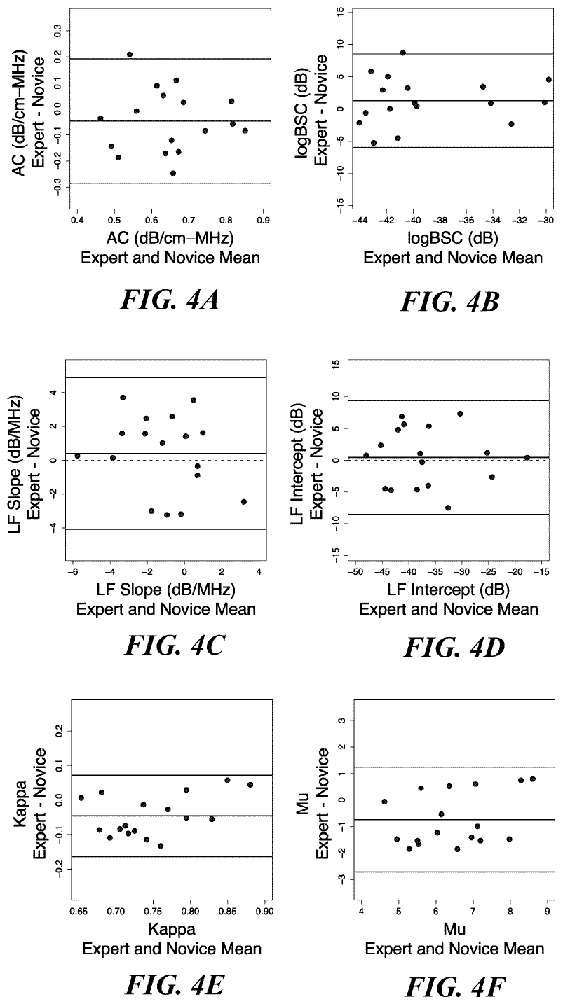

FIGS. 4A-4F show BA plots, obtained in the example study, for paired expert sonographer and novice operator measurements for each QUS parameter. In FIGS. 4A-4F, the horizontal lines on each plot represent bias and 95% limits of agreement. The Y-axis on each BA plot was scaled by wCV to facilitate visual comparison of relative agreement across plots, except for the plot for LF slope, where the Y-axis was scaled to accommodate spread of the data. As shown in FIGS. 4A-4F, ICC ranged from 0.54 (AC) to 0.86 (LF Intercept). wCV ranged from 6.8% (logBSC) to 15% (AC), with the exception of LF Slope, which could not be computed because the measurements overlapped zero. Bland-Altman bias was significant for Kappa and Mu, but not significant for AC, BSC, LF Intercept or LF Slope.

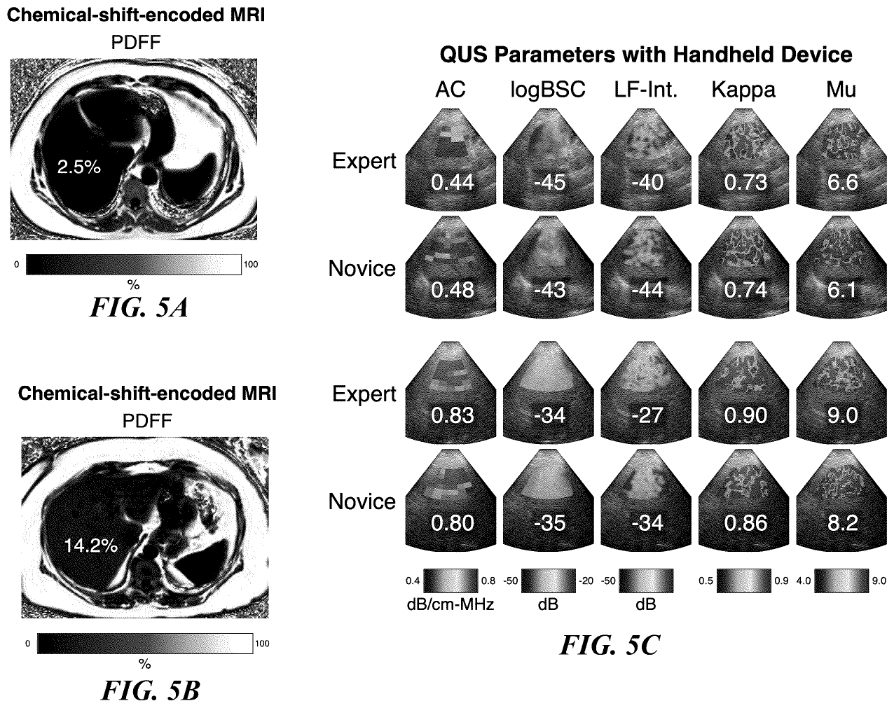

FIG. 5C shows representative QUS parametric maps (AC, log BSC, LF intercept, Kappa, Mu) acquired in two participants, one with low PDFF (2.5%) and one with elevated PDFF (14.2%), along with the corresponding PDFF maps (FIGS. 5A-5B). For each participant, two sets of QUS parametric color maps are displayed in FIG. 5C (AC, logBSC, Lizzi-Feleppa (LF) intercept, Kappa, and Mu) with overlain parameter values, one set obtained by an expert (first row) and the other set by a novice (second row). As shown in FIG. 5C, there is close agreement in QUS values by expert and novice operators in both participants. Also shown in FIG. 5C, QUS values are more positive (or less negative) in the participant with mild steatosis than in the participant without steatosis.

Results of the example study described above demonstrate that QUS with POCUS, in accordance with disclosed techniques, is feasible in adults with overweight or obesity and can be performed by either expert or novice operators after limited training. The diagnostic classification of fatty liver was accurate for both expert and novice operators, when using QUS data from the POCUS device. Five of the six QUS parameters were moderately and significantly correlated with

MRI PDFF, regardless of the operator. Expert sonographers did slightly better than novice operators in comparison to ground truth for diagnosis and quantification of liver fat. Reproducibility between expert sonographers and novice operators was moderate to excellent, varying with the QUS parameter.

The clinical studies on QUS were obtained from a POCUS device or using a CMUT transducer to assess fatty liver disease. The correlation between individual QUS parameters acquired with a POCUS device and reference PDFF, regardless of POCUS operator expertise, are comparable to those reported previously using full-size US systems operated by expert sonographers and with controlled attenuation parameter performed by trained operators.

Additionally, the AUC for diagnosis of hepatic steatosis using QUS acquired on a POCUS device by expert sonographers or novice operators were comparable to those reported previously for BSC or UGAP acquired by expert sonographers with full-size systems, and were higher than those achieved with controlled attenuation parameter. The inter-operator ICCs for AC and BSC between expert and novice operators were slightly lower than those reported previously between expert sonographers using full-size systems.

The effectiveness of the disclosed techniques to assess liver fat was exemplified in the example study. The promising performance feasibility of QUS on an inexpensive handheld POCUS device has important implications. The low cost and compact size of the device makes it straightforward to deploy in resource limited settings. Moreover, the device for this application can be operated by novices with minimal training, which would be necessary to facilitate widespread deployment. This type of technology could become widely disseminated to screen for fatty liver disease in community clinics, mobile health, and remote or disadvantaged areas, which is potentially transformative by increasing access to early diagnosis with its potential to prevent disease progression.

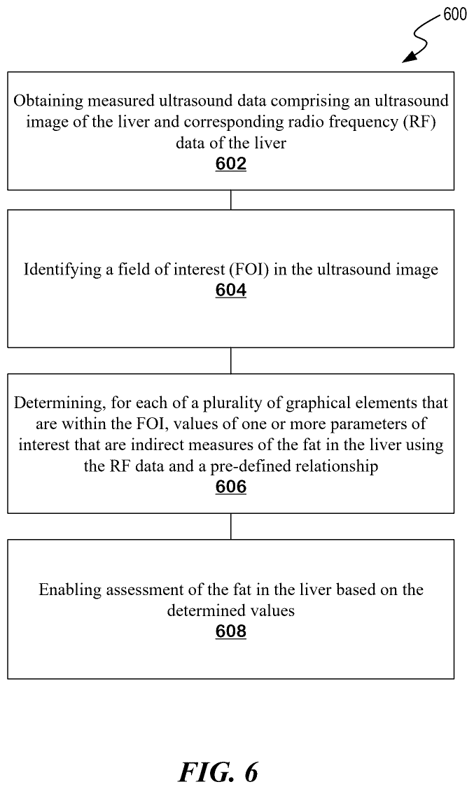

FIG. 6 shows a flowchart of an example method 600 for quantitatively assessing fat of a liver. At operation 602, the method 600 comprises obtaining measured ultrasound data comprising an ultrasound image of the liver and corresponding radio frequency (RF) data of the liver. At operation 604, the method 600 comprises identifying a field of interest (FOI) in the ultrasound image. At operation 606, the method 600 comprises determining, for each of a plurality of graphical elements that are within the FOI, values of one or more parameters of interest that are indirect measures of the fat in the liver using the RF data and a pre-defined relationship. At operation 608, the method 600 comprises enabling assessment of the fat in the liver based on the determined values.

Implementations of the subject matter and the functional operations described in this patent document can be implemented using data processing units that include various systems, digital electronic circuitry, or in computer software, firmware, or hardware, including the structures, modules and components disclosed in this specification and their structural equivalents, or in combinations of one or more of them. Implementations of the subject matter pertaining to data processing described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit”, “data processing module”, or “data processing apparatus”, or the like, encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

1. A method for quantitatively assessing fat of a liver, comprising:
obtaining measured ultrasound data comprising an ultrasound image of the liver and corresponding radio frequency (RF) data of the liver;
identifying a field of interest (FOI) in the ultrasound image;
determining, for each of a plurality of graphical elements that are within the FOI, values of one or more parameters of interest that are indirect measures of the fat in the liver using the RF data and a pre-defined relationship; and
enabling assessment of the fat in the liver based on the determined values.

2. The method of claim 1, further comprising generating a liver fat deposition map showing deposition of the fat in the liver by assigning, to each of the graphical elements, a color associated with the values.

3. The method of claim 2, wherein the liver fat deposition map is two-dimensional or three-dimensional, and each of the plurality of graphical elements is a pixel or voxel.

4. The method of claim 2, further comprising superimposing the liver fat deposition map over the ultrasound image to enable visualization of one or more of the values at locations within the ultrasound image.

5. The method of claim 1, wherein the ultrasound data is acquired using a point-of-care ultrasound (POCUS) device.

6. The method of claim 1, wherein the pre-defined relationship is determined by:
for each of a plurality of sample subjects, obtaining a measured ground truth data and sample ultrasound data of the sample subject, wherein the measured ground truth data of the sample subject comprises a distribution of liver fat metrics, wherein each of the liver fat metrics in the distribution is related to at least one of the one or more parameters; and
determining the pre-defined relationship based on the measured ground truth data and the sample ultrasound data of the plurality of sample subjects.

7. The method of claim 6, wherein the measured ground truth data comprises magnetic resonance imaging (MRI).

8. The method of claim 1, wherein the one or more parameters of interest comprise one or more of an attenuation coefficient, a backscatter coefficient, a Lizzi-Feleppa slope, a Lizzi-Feleppa intercept, a Kappa, or a Mu.

9. The method of claim 1, comprising determining a value of the fat in the liver for each of the graphical elements based on the values of the one or more parameters of interest of the graphical element or a combination of the values of the one or more parameters of interest of the graphical element.

10. The method of claim 1, wherein the method is implemented to provide a diagnosis of the liver or a grading of fat in the liver.

11. A system for quantitatively assessing fat of a liver, comprising a point-of-care ultrasound (POCUS) device configured to obtain ultrasound data comprising an ultrasound image of the liver and corresponding radio frequency (RF) data of the liver; and
one or more processors configured to receive the ultrasound data from the POCUS device and to:
identify a field of interest (FOI) in the ultrasound image, determine, for each of a plurality of graphical elements that are within the FOI, values of one or more parameters of interest that are indirect measures of the fat in the liver using the RF data and a pre-defined relationship,
generate a liver fat deposition map by assigning, to each of the graphical elements, a color associated with the values, and
enable display of the liver fat deposition map to allow assessment of the fat in the liver based on the determined values.

12. The system of claim 11, wherein the liver fat deposition map is two-dimensional or three-dimensional, and each of the plurality of graphical elements is a pixel or voxel.

13. The system of claim 11, wherein the pre-defined relationship is determined by:
for each of a plurality of sample subjects, obtaining a measured ground truth data and sample ultrasound data of the sample subject, wherein the measured ground truth data of the sample subject comprises a distribution of liver fat metrics, wherein each of the liver fat metrics in the distribution is related to at least one of the one or more parameters; and
determining the pre-defined relationship based on the measured ground truth data and the sample ultrasound data of the plurality of sample subjects.

14. The system of claim 13, wherein the one or more processors are further configured to receive the measured ground truth data from one or more additional processors or devices, wherein the measured ground truth data comprises magnetic resonance imaging (MRI) or biopsy data.

15. The system of claim 11, wherein the one or more parameters of interest comprise one or more of an attenuation coefficient, a backscatter coefficient, a Lizzi-Feleppa slope, a Lizzi-Feleppa intercept, or a Kappa, or a Mu.

16. The system of claim 11, wherein the one or more processors are further configured to:
determine a value of the fat in the liver, for each of the graphical elements, based on the values of the one or more parameters of interest of the graphical element or combinations of the values of the one or more parameters of interest of the graphical element.

17. A computer program product having code stored thereon, the code, when executed by a processor, causing the processor to implement a method for quantitatively assessing fat of a liver, comprising:
obtaining measured ultrasound data comprising an ultrasound image of the liver and corresponding radio frequency (RF) data of the liver;
identifying a field of interest (FOI) in the ultrasound image, determining, for each of a plurality of graphical elements that are within the FOI, values of one or more parameters of interest that are indirect measures of the fat in the liver using the RF data and a pre-defined relationship; and
enabling assessment of the fat in the liver based on the determined values.

18. The computer program product of claim 17, wherein the method further comprises generating a liver fat deposition map showing deposition of the fat in the liver by assigning, to each of the graphical elements, a color associated with the values, wherein the liver fat deposition map is two-dimensional or three-dimensional, and each of the plurality of graphical elements is a pixel or voxel.

19. The computer program product of claim 17, wherein the pre-defined relationship is determined by:
for each of a plurality of sample subjects, obtaining a measured ground truth data and sample ultrasound data of the sample subject, wherein the measured ground truth data of the sample subject comprises a distribution of liver fat metrics, wherein each of the liver fat metrics in the distribution is related to at least one of the one or more parameters; and
determining the pre-defined relationship based on the measured ground truth data and the sample ultrasound data of the plurality of sample subjects.

20. The computer program product of claim 17, wherein the ultrasound data is acquired using a point-of-care ultrasound (POCUS) device.
